(19) United States
(12) Patent Application Publication
(10) Pub. No.: US 2021/0330355 A1
(43) Pub. Date: Oct. 28, 2021

(54) PUNCTURE DEVICE ASSOCIATED WITH MEDICAL ASSEMBLY

(71) Applicant: Baylis Medical Company Inc., Montreal (CA)

(72) Inventors: Jackie Leung, Richmond Hill (CA); Eduardo Moriyama, Richmond (CA); Gareth Davies, Toronto (CA); Kaylie Lau, Toronto (CA)

(21) Appl. No.: 17/231,077

(22) Filed: Apr. 15, 2021

Related U.S. Application Data
(60) Provisional application No. 63/013,617, filed on Apr. 22, 2020.

(51) Int. Cl. A61B 17/34; A61B 17/00

(52) U.S. Cl. CPC A61B 17/3478; A61B 2017/00247; A61B 17/00234

(57) ABSTRACT

An elongated medical assembly has a distal-assembly tip. The distal-assembly tip is, at least in part, selectively maneuverable along an assembly-tip movement path terminating proximate to a first biological feature of a biological structure of a patient. An elongated puncture device has a distal puncture tip. The distal puncture tip is, at least in part, selectively deployable along a puncture-tip movement path extending from the elongated medical assembly and into, at least in part, the first biological feature, and also extending from the first biological feature toward a second biological feature of the patient.

### TECHNICAL FIELD

This document relates to the technical field of (and is not limited to) a synergistic combination of an elongated medical assembly and an elongated puncture device (and method therefor); and this document also relates to the technical field of (and is not limited to) an elongated puncture device for an elongated medical assembly (and method therefor).

### BACKGROUND

Known medical devices are configured to facilitate a medical procedure, and/or help healthcare providers diagnose and/or treat medical conditions of sick patients, etc.

### SUMMARY

It will be appreciated that there exists a need to mitigate (at least in part) at least one problem associated with the existing (known) medical assemblies (also called the existing technology). After much study of, and experimentation with, the existing (known) medical assemblies, an understanding (at least in part) of the problem and its solution have been identified (at least in part) and are articulated (at least in part) as follows:

Laceration (an elongated cut) of the valve leaflet (of the heart of a patient) is a preventative procedure performed to minimize the risk of obstruction of the right coronary artery (RCA) of the heart after the transcatheter replacement of an aortic valve. The procedure first may require the puncture and traversal of the valve leaflet with a puncturing device (such as a wire, etc.). The puncture location (to be formed on the valve leaflet) may be a critical factor in the outcome of the procedure. Furthermore, laceration of the valve leaflet may permanently impair the valve function so this case may represent a point of no return for the transcatheter aortic valve replacement (TAVR) procedure.

It may be desirable to provide a deviation to the known (current) BASILICA and LAMPOON practice (procedure). As an alternative to pre-emptively lacerating the valve leaflet, a medical apparatus may be positioned such that the valve leaflet may be punctured (perforated) after a replacement valve (known and not depicted and further described) is implanted. In this manner, the laceration valve leaflet may begin from the puncture location on the valve leaflet (by a motion of the puncturing device via the right coronary artery), resulting in a relatively lower possibility (preferably, no possibility) of inadvertent damage to the valve leaflet (before the replacement valve is, advantageously, positioned for installation in the heart).

In accordance with a preferred embodiment, there is provided a puncture device (such as, a radio frequency wire device) configured to form a degree bend once the puncture device is positioned (at least in part) inside the right coronary artery (RCA) of the heart; this is initially done, preferably, in such a way that a distal tip of the puncture device becomes spatially oriented (outwardly) from the RCA of the heart towards the heart chamber. After the valve implant (not depicted but known) is deployed, the puncture device (also called a lacerating device) may be withdrawn from the RCA toward the obstructing valve leaflet;

then, a puncturing distal tip (such as, an radio-frequency electrode positioned at the puncturing distal tip) of the puncture device is used (activated) to puncture the valve leaflet obstructing the right coronary artery. The puncture device, which now protrudes from the puncture site (formed through the valve leaflet) may be snared and pulled, etc. For instance, the cutting tip (such as, an electrode) of the puncture device may be positioned or situated at the degree bend, and then may be activated (energized) to carry out the formation of the puncture hole (laceration) to be formed through the leaflet, similar to the BASILICA and LAMPOON practice (procedure).

It will be appreciated that the apparatus may be utilized for the prevention of occlusion of the right coronary artery after implementation of the TAVR procedure.

To mitigate, at least in part, at least one problem associated with the existing technology, there is provided (in accordance with a major aspect) an apparatus. The apparatus includes and is not limited to (comprises) a synergistic combination of an elongated medical assembly and an elongated puncture device. The elongated medical assembly has a distal-assembly tip. The distal-assembly tip is, at least in part, selectively maneuverable (configured to be selectively maneuverable) along an assembly-tip movement path terminating proximate to a first biological feature (of a biological structure of a patient). The elongated puncture device has a distal puncture tip. The distal puncture tip is, at least in part, selectively deployable (configured to be selectively deployable) along a puncture-tip movement path (a movement axis) extending from the elongated medical assembly and into, at least in part, the first biological feature, and then extending from the first biological feature toward a second biological feature of the patient.

To mitigate, at least in part, at least one problem associated with the existing technology, there is provided (in accordance with a major aspect) an apparatus. The apparatus is utilized with (is configured to be utilized with) an elongated medical assembly.

The elongated medical assembly has a distal-assembly tip. The distal-assembly tip is, at least in part, selectively maneuverable (configured to be selectively maneuverable) along an assembly-tip movement path terminating proximate to a first biological feature (of a biological structure of a patient). The apparatus includes and is not limited to (comprises) an elongated puncture device. The elongated puncture device has a distal puncture tip. The distal puncture tip is, at least in part, selectively deployable (configured to be selectively deployable or maneuverable) along a puncture-tip movement path extending from the elongated medical assembly and into, at least in part, the first biological feature, and then extending from the first biological feature toward a second biological feature of the patient.

To mitigate, at least in part, at least one problem associated with the existing technology, there is provided (in accordance with a major aspect) a method. The method is for operating an elongated medical assembly having a distal-assembly tip and an elongated puncture device having a distal puncture tip. The method includes and is not limited to (comprises) selectively maneuvering, at least in part, the distal-assembly tip, of the elongated medical assembly, along an assembly-tip movement path terminating proximate to a first biological feature (of a biological structure of a patient). The method also includes and is not limited to selectively deploying, at least in part, the distal puncture tip, of the elongated puncture device, along a puncture-tip movement path extending from the elongated medical assembly and into, at least in part, the first biological feature, and then extending from the first biological feature toward a second biological feature of the patient.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1, FIG. 2, FIG. 3, FIG. 4 and FIG. 5 depict side views of embodiments of an elongated medical assembly and an elongated puncture device.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

LISTING OF REFERENCE NUMERALS USED IN THE DRAWINGS

medical assembly 102
distal-assembly tip 103
exit portal 104
puncture site 105
puncture device 106
distal puncture tip 108
medical sheath assembly 202
distal exit section 204
lumen 206
elongated member 301
elongated member 302
assembly-tip movement path 404
puncture-tip movement path 406
obtuse angle 408
bent section 410
tip-snare device 500
snare-retrieval path 502
first cutting element 601
second cutting element 602
laceration 700
patient 900
biological structure 902
heart 903
first biological feature 911
second biological feature 912
right coronary artery 921
valve leaflet 922

### DETAILED DESCRIPTION OF THE EXEMPLARY EMBODIMENTS

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

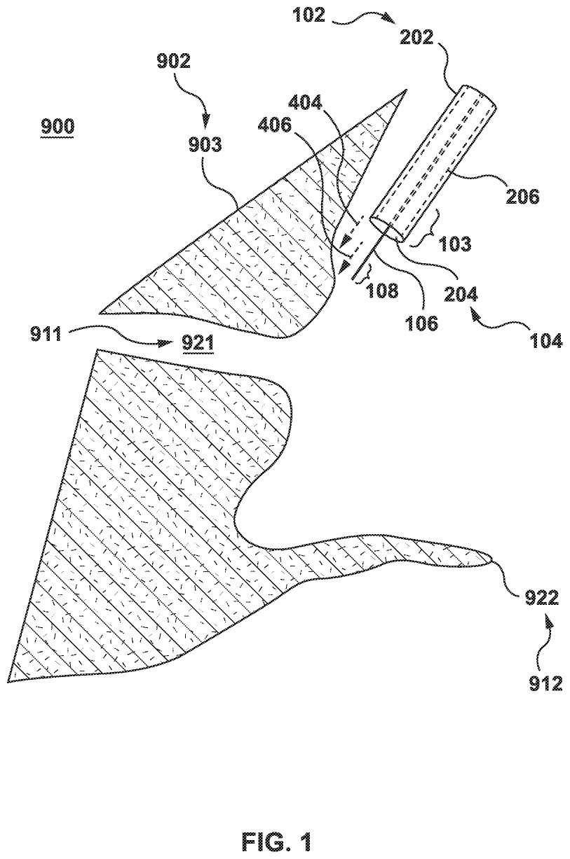

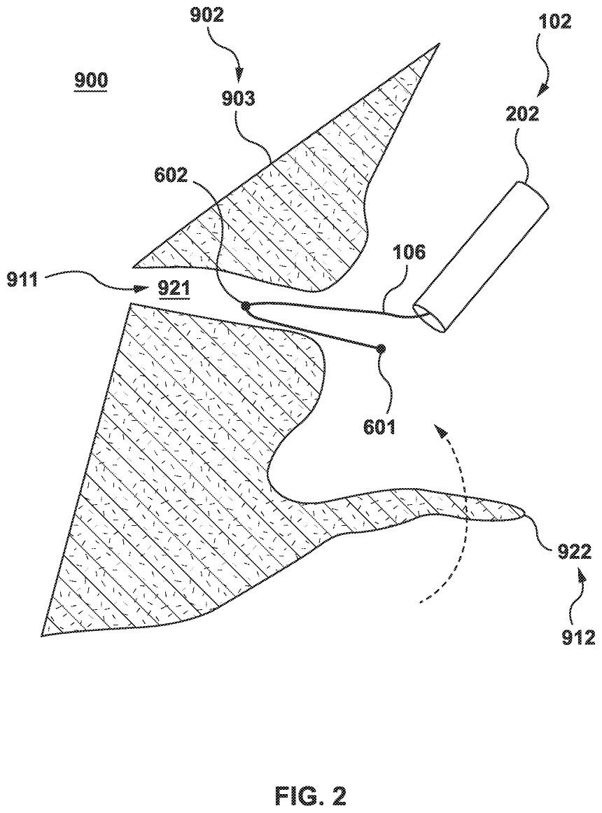

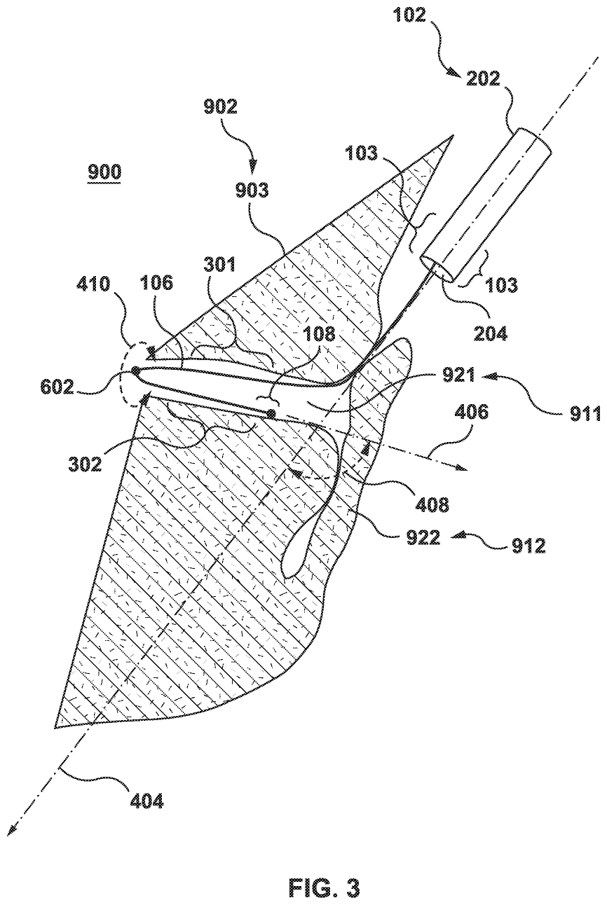

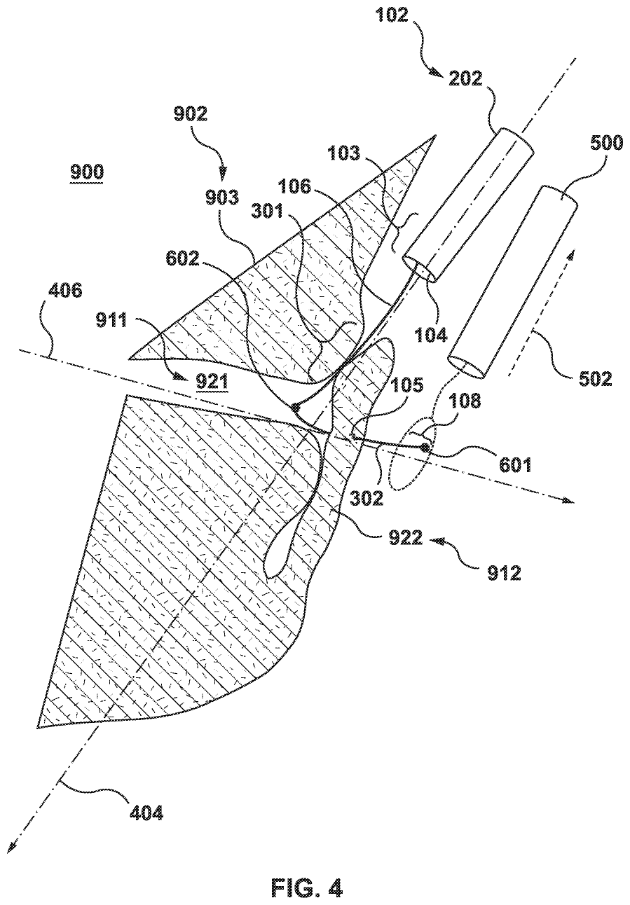

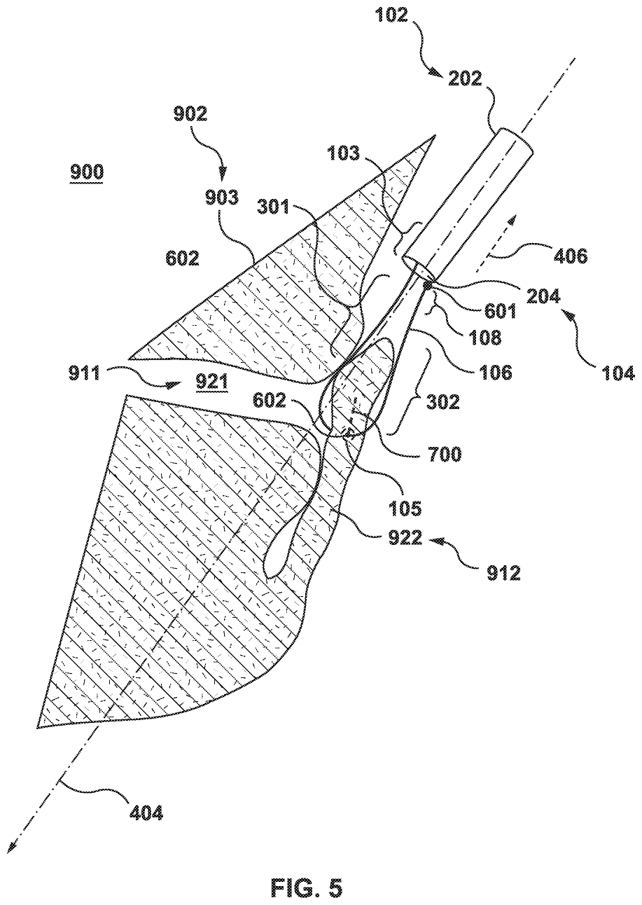

FIG. 1, FIG. 2, FIG. 3, FIG. 4 and FIG. 5 depict side views of embodiments of an elongated medical assembly 102 and an elongated puncture device 106.

Referring to the embodiment as depicted in FIG. 1, the elongated medical assembly 102 is, at least in part, selectively maneuverable (configured to be selectively maneuverable) along an assembly-tip movement path 404. The elongated medical assembly 102 remains relatively stationary after being positioned as depicted in FIG. 1. Generally, the elongated medical assembly 102 includes any such medical assembly configured to be selectively maneuverable, at least in part, along a confined space of the patient 900. The assembly-tip movement path 404 terminates (at a position located) proximate to a first biological feature 911 of a biological structure 902 of a patient 900. The elongated puncture device 106 is configured to be positioned in (and ready or on standby, for deployment from) the elongated medical assembly 102. In accordance with a preferred embodiment (depicted in FIG. 1), the elongated puncture device 106 may be placed in a straightened condition (substantially without any significant bent section or bent sections) inside the elongated medical assembly 102 (such as a sheath assembly, etc.). In accordance with preferred embodiments (as depicted in FIG. 2, FIG. 3, FIG. 4 and FIG. 5), the elongated puncture device 106 is deployed from the elongated medical assembly 102, and then the elongated puncture device 106, in use, takes on a bent shape (after the elongated puncture device 106 is advanced outwardly from the elongated medical assembly 102); it will be appreciated that the elongated puncture device 106 may be loaded into the elongated medical assembly 102 while the elongated puncture device 106 is ins a bent condition (if so desired); the elongated puncture device 106 may include, for instance, a memory shape alloy for achieving such an arrangement, etc. The elongated puncture device 106 is configured to be deployed from the elongated medical assembly 102; this is preferably done after, or once, the elongated medical assembly 102 is, at least in part, maneuvered proximate to the first biological feature 911, as depicted in FIG. 3. The distal puncture tip 108 (of the elongated puncture device 106) is selectively maneuverable, and extendable from, at least in part, the elongated medical assembly 102, along the puncture-tip movement path 406. As depicted in FIG. 1, the distal puncture tip 108 of the elongated puncture device 106 is positioned or located (in a stowage condition or a standby condition) proximate to the distal-assembly tip 103 of the elongated medical assembly 102.

Referring to the embodiments as depicted in FIG. 1 and FIG. 2, the second biological feature 912 (or the valve leaflet 922) is spaced apart from the opening leading into the first biological feature 911 (or the right coronary artery 921). Referring to the embodiment as depicted in FIG. 1, the elongated puncture device 106 is initially deployed from the elongated medical assembly 102 (or the medical sheath assembly 202). Referring to the embodiment as depicted in FIG. 2, the elongated puncture device 106 is deployed, at least in part, and extends into the right coronary artery 921 (the first biological feature 911) while the valve leaflet 922 remains spaced apart from the opening leading into the first biological feature 911 (in this manner, the valve leaflet 922 is in a valve open condition). Referring to the embodiments as depicted in FIG. 3 to FIG. 5, the second biological feature 912 (or the valve leaflet 922) is positioned against (and remains positioned against) the opening leading into the first biological feature 911 (or the right coronary artery 921), and in this manner, the valve leaflet 922 is in a valve closed condition.

Referring to the embodiment as depicted in FIG. 1, the elongated medical assembly 102 is (preferably) configured to guide the insertion of an elongated puncture device 106 and/or any equivalent thereof into the confined space defined by the patient 900. The elongated medical assembly 102 includes, preferably, a flexible tube (made from a medical grade material). The elongated medical assembly 102 is (preferably) impermeable by any bodily fluids. The elongated medical assembly 102 includes (in accordance with a preferred embodiment) bio-compatible materials properties suitable for sufficient performance properties (dielectric strength, thermal performance, insulation and corrosion, water and heat resistance) for safe performance to comply with industrial and regulatory safety standards (or compatible for medical usage). Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]:Elsevier/William Andrew, [2014].

Referring to the embodiment as depicted in FIG. 1, the elongated puncture device 106 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus is applied to the shape-memory material. This is known as the shape memory effect (SME). Superelasticity (in alloys) may be observed once the shape-memory material is deformed under the presence (an application) of a stimulus force. The elongated puncture device 106 includes (in accordance with another option) superelastic nitinol. Nitinol alloys exhibit two closely related and unique properties: shape memory effect (SME) and superelasticity (SE; also called pseudoelasticity or PE). Shape memory is the ability of nitinol to undergo deformation at one temperature, then recover its original, undeformed shape upon heating above its transformation temperature. Superelasticity occurs at a narrow temperature range just above its transformation temperature; in this case, no heating is necessary to cause the undeformed shape to recover, and the material exhibits enormous elasticity, from about ten (10) to thirty (30) times that of ordinary metal.

Referring to the embodiment as depicted in FIG. 1, the biological structure 902 incudes (by way of example and is not limited to) the heart 903 of the patient 900. The first biological feature 911 includes (and is not limited to) the right coronary artery 921 (RCA) of the heart 903.

The second biological feature 912 includes (and is not limited to) a valve leaflet 922 of the heart 903.

Referring to the embodiment as depicted in FIG. 1, the elongated medical assembly 102 includes an exit portal 104, in which the assembly-tip movement path 404 is extendable, at least in part, from the exit portal 104. The elongated puncture device 106 is selectively maneuverable, at least in part, away from the exit portal 104 of the elongated medical assembly 102.

Referring to the embodiment as depicted in FIG. 1, the elongated medical assembly 102 includes a medical sheath assembly 202. The medical sheath assembly 202 has a distal exit section 204 leading to a lumen 206 defined along, at least in part, a length of the medical sheath assembly 202. The elongated puncture device 106 is configured to be receivable, and movable, along, at least in part, the lumen 206 of the medical sheath assembly 202.

Referring to the embodiment as depicted in FIG. 3, the elongated medical assembly 102 continues to remain positioned (relatively) proximate to the first biological feature 911 (of the biological structure 902). This is done, preferably, while the distal puncture tip 108 (of the elongated puncture device 106) is deployed from the elongated medical assembly 102. In this manner, inadvertent injury to the patient 900 may be mitigated (reduced, at least in part). The elongated medical assembly 102 is maintained at a relatively stationary condition or position; this is done, preferably, in such a way that the distal-assembly tip 103 of the elongated medical assembly 102 is not substantially maneuvered (during this procedure, for example) along the assembly-tip movement path 404. The elongated medical assembly 102 is, preferably held (or maintained) in a relatively stationary condition; this is done, preferably, so that the elongated medical assembly 102 does not substantially move relative to the biological structure 902, and the elongated puncture device 106 may then be deployed in a relatively safer manner, preferably without, or minimizing, inadvertent injury to the patient 900. The elongated puncture device 106 is deployed (from the standby condition, as depicted in FIG. 1) from (within an interior of) the elongated medical assembly 102; this is preferably done after, or once, the elongated medical assembly 102 is, at least in part, maneuvered proximate to the first biological feature 911. The distal puncture tip 108 is, at least in part, selectively deployed (maneuvered) along the puncture-tip movement path 406. The assembly-tip movement path 404 is aligned (or is alignable), at least in part, into (and along a part of) the first biological feature 911 (such as into the interior of the first biological feature 911). The puncture-tip movement path 406 extends from the elongated medical assembly 102 and into, at least in part, (the interior of) the first biological feature 911. The puncture-tip movement path 406 further extends from the first biological feature 911 toward a second biological feature 912 of the patient 900 (as depicted in FIG. 4).

Referring to the embodiment as depicted in FIG. 3, the elongated puncture device 106 includes a first cutting element 601 and a second cutting element 602. The second cutting element 602 is spaced apart from the first cutting element 601. The first cutting element 601 is positioned at (a front end of) the distal puncture tip 108. The first cutting element 601 is configured to form the puncture site 105 through the second biological feature 912 (once the elongated puncture device 106 is moved and activated accordingly). The first cutting element 601 may include, for instance, a first electrode portion configured to be electrically actuated, such as with an amount of radio frequency energy of sufficient magnitude for cutting tissue.

Generally, the first cutting element 601 is, preferably, configured to deliver an amount of radio frequency energy that is sufficient for cutting soft tissue. The second cutting element 602 is positioned on a section (portion) of the elongated puncture device 106, and is set back from the first cutting element 601. The second cutting element 602 may be similar to the first cutting element 601. The second cutting element 602 may include, for instance, a second electrode portion configured to be electrically actuated, such as with an amount of radio frequency energy of sufficient magnitude for cutting soft tissue. Generally, the second cutting element 602 is (preferably) configured to deliver and amount of radio frequency energy that is sufficient enough for cutting soft tissue. The second cutting element 602 is positioned to cut (lacerate or form the laceration 700 extending along) the second biological feature 912 (or the valve leaflet 922) after (A) the first cutting element 601 forms the puncture site 105 through the second biological feature 912, and (B) the first cutting element 601 is further moved beyond the puncture site 105). The second cutting element 602 is configured to cut the second biological feature 912 starting from the puncture site 105 (after the second cutting element 602 is positioned in the puncture site 105 formed in the second biological feature 912). For instance, the second cutting element 602 may be positioned at an outer surface of the elongated puncture device 106 at the bent section 410 (such as, a 180 degree bend) formed in the elongated puncture device 106 (as depicted in FIG. 3).

Referring to the embodiment as depicted in FIG. 3, the second cutting element 602 is positioned in the right coronary artery 921 (the first biological feature 911) with the first cutting element 601 pointing (facing) outwardly from the right coronary artery 921 toward the valve leaflet 922 (the second biological feature 912). The valve leaflet 922 has already been pushed against and opening leading to the right coronary artery 921 (this is done in such a way that the valve leaflet 922 is obstructing the blood flow through the right coronary artery 921).

Referring to the embodiment as depicted in FIG. 4, the elongated medical assembly 102 is maintained at a relatively stationary condition or position. This is preferably done in such a way that the distal-assembly tip 103 (of the elongated medical assembly 102) is not maneuvered along the assembly-tip movement path 404 (while the distal puncture tip 108 is maneuvered, at least in part, along the puncture-tip movement path 406; this is done to mitigate potential patient injury). The distal puncture tip 108 (of the elongated puncture device 106) continues to be selectively deployed (maneuvered) along the puncture-tip movement path 406 extending from (within) the first biological feature 911 toward (a position located proximate to) the second biological feature 912 of the patient 900. The distal puncture tip 108 (of the elongated puncture device 106) is further deployed (activated or maneuvered) to form a puncture site 105. The puncture site 105 extends through the second biological feature 912. This is preferably done once the elongated puncture device 106 is deployed (maneuvered), at least in part, along the puncture-tip movement path 406, and the elongated puncture device 106 is located at (positioned proximate to) the second biological feature 912. It will be appreciated that the elongated puncture device 106 is configured to form the puncture site 105 in any suitable manner. For instance, the elongated puncture device 106 may include a radio frequency wire with a radio frequency electrode fixedly positioned at the distal puncture tip 108. The elongated puncture device 106 may include (and is not limited to) a radio frequency puncture device, such as the BAYLIS (TRADEMARK) POWERWIRE (REGISTERED TRADEMARK) radio frequency guidewire manufactured by BAYLIS MEDICAL COMPANY (headquartered in Canada). In accordance with another embodiment, the elongated puncture device 106 includes (and is not limited to) an elongated guidewire having a distal tip section presenting a mechanical cutting portion (for this case, the puncture site 105 is formed by physically moving the mechanical cutting portion into the second biological feature 912). Once the distal puncture tip 108 (of the elongated puncture device 106) has formed the puncture site 105 (through the second biological feature 912), the elongated puncture device 106 may be maneuvered (or further deployed to move) away from puncture site 105 (and preferably toward) the elongated medical assembly 102. For instance, once the distal puncture tip 108 protrudes (and extends), at least in part, beyond the puncture site 105, the distal puncture tip 108 may be (securely) snared (as depicted in FIG. 4) and then pulled away (dragged) from the puncture site 105 (preferably toward the elongated medical assembly 102). For instance, a tip-snare device 500 is configured to be maneuvered proximate to the distal puncture tip 108; this is done in such a way that the tip-snare device 500, in use, snares, and then the tip-snare device 500 (in use) is moved to take (pull or drag) the distal puncture tip 108 along a snare-retrieval path 502, etc.

Referring to the embodiment as depicted in FIG. 4, the distal puncture tip 108 is snared (by operation of the tip-snare device 500 or any equivalent thereof) after the distal puncture tip 108 (or the first cutting element 601) has punctured (formed the puncture site 105 through) the valve leaflet 922 (and extend therefrom), which is then followed by the formation of a laceration 700 (an elongated cut) during a laceration procedure, to be performed by movement of the second cutting element 602 from the puncture site 105 and along a length of the valve leaflet 922, as the tip-snare device 500 and the elongated puncture device are concurrently withdrawn away from the puncture site 105 (as described in the BASILICA procedure or LAMPOON procedure).

Referring to the embodiment as depicted in FIG. 5, the distal puncture tip 108 is configured to protrude and extend, at least in part, beyond the puncture site 105. The distal puncture tip 108 is also configured to be maneuvered away from the puncture site 105 (preferably toward the elongated medical assembly 102). For instance, the elongated puncture device 106 may include a memory shape metal configured to facilitate such described and depicted maneuvered movements of the elongated puncture device 106. The laceration 700 is formed by movement of the second cutting element 602 starting from the puncture site 105 and along a length of the valve leaflet 922, as depicted in FIG. 5; this action is performed after the first cutting element 601 has formed the puncture site 105 and the elongated puncture device 106 and the distal puncture tip 108 are moved accordingly away from the valve leaflet 922). The second cutting element 602 is configured to form the laceration 700 along, at least in part, the second biological feature 912 starting from the puncture site 105 (once the elongated puncture device 106 is moved accordingly away from the puncture site 105).

Referring to the embodiment as depicted in FIG. 5, there is depicted an alternative embodiment in which the elongated puncture device 106 has a stiffness attribute configured to become resiliently bent enough (to a degree) such that the tip-snare device 500 (as depicted in FIG. 4) is not required to perform laceration operation (lacerating or cutting action) with the second cutting element 602. The second cutting element 602 is positioned in the puncture site 105, and the distal puncture tip 108 forms a natural bent section (as depicted), and then the elongated medical assembly 102 is retracted (along with the elongated puncture device 106) away from the valve leaflet 922 along the puncture-tip movement path 406 (the elongated medical assembly 102 is retracted away from the valve leaflet 922). The laceration (elongated cut or the laceration 700) is then linearly formed along the valve leaflet 922 by the second cutting element 602. An amount of radio frequency energy may be delivered (conveyed) to the first cutting element 601 and the second cutting element 602 via an energy generator (known and not depicted or described) configured to be connected at the proximal end of the elongated puncture device 106, and preferably controlled via a switch by a user, etc. The first cutting element 601 is configured to be (once energized) to perforate (form the puncture site 105 through) the valve leaflet 922 (with the first cutting element 601 moving toward the valve leaflet 922 from a position located in the right coronary artery 921). After the valve leaflet 922 is perforated by the first cutting element 601, the second cutting element 602 is energized (and moved) to lacerate the valve leaflet 922 (that is, for the formation of the laceration 700, as depicted in FIG. 5). For instance, the elongated puncture device 106 may be configured to be steerable (such as by using a steering mechanism, known and not depicted), and/or utilizing a memory shape metal, etc.). In accordance with an alternative embodiment, the second cutting element 602 does not exist (is not provided), and the operator may retract the elongated puncture device 106 (after the elongated puncture device 106 is snared) thereby manually removing a portion of an electrical insulation from a surface of the elongated puncture device 106, and the exposed outer portion of the elongated puncture device 106 is then configured to act as a lacerating electrode (which is equivalent to the second cutting element 602), similar to the arrangement performed in the BASILICA or LAMPOON procedure (known and not depicted or described in any detail). Other options may include the following: (A) flushing with non-ionic fluid to insulate the bend electrode (that is, the second cutting element 602) during perforation of (formation of the puncture hole through) the valve leaflet 922; (B) incorporating nitinol or other shape-memory material to maintain the profile of the elongated puncture device 106; and/or (C) stiffening and shaping the bent section 410 (as depicted in FIG. 3) such that snaring of the elongated puncture device 106 may be performed similarly to the BASILICA or LAMPOON procedure (which may not be necessary).

Referring to the embodiments as depicted in FIG. 1, FIG. 3 and FIG. 4, an apparatus is depicted. The apparatus includes and is not limited to a synergistic combination of an elongated medical assembly 102 and an elongated puncture device 106. The elongated medical assembly 102 may include a medical sheath assembly, and any equivalent thereof. The elongated medical assembly 102 has a distal-assembly tip 103. The distal-assembly tip 103 is, at least in part, selectively maneuverable along an assembly-tip movement path 404. The assembly-tip movement path 404 may be called a movement axis or a movement direction, etc., and any equivalent thereof. The assembly-tip movement path 404 terminates (is located or positioned) proximate to a first biological feature 911 (of a biological structure 902 of a patient 900). The elongated puncture device 106 has a distal puncture tip 108. The distal puncture tip 108 may be called a puncture-tip portion. The distal puncture tip 108 is, at least in part, selectively deployable (selectively maneuverable) along a puncture-tip movement path 406. The puncture-tip movement path 406 may be called a movement axis or a movement direction. The puncture-tip movement path 406 extends from the elongated medical assembly 102 and into, at least in part, the first biological feature 911. The puncture-tip movement path 406 also extends from the first biological feature 911 toward a second biological feature 912 of the patient 900.

Referring to the embodiments as depicted in FIG. 1, FIG. 3 and FIG. 4, there is depicted an apparatus. The apparatus is utilized with (is configured to be utilized with) an elongated medical assembly 102. The elongated medical assembly 102 has a distal-assembly tip 103. The distal-assembly tip 103 is, at least in part, selectively maneuverable along an assembly-tip movement path 404 terminating proximate to the first biological feature 911 of the biological structure 902 of the patient 900. The apparatus includes, and is not limited to, an elongated puncture device 106 having a distal puncture tip 108. The distal puncture tip 108 is, at least in part, selectively deployable (selectively maneuverable) along a puncture-tip movement path 406. The puncture-tip movement path 406 extends from the elongated medical assembly 102 and into, at least in part, the first biological feature 911. The puncture-tip movement path 406 also extends from the first biological feature 911 toward a second biological feature 912 of the patient 900.

Referring to the embodiments as depicted in FIG. 1, FIG. 3 and FIG. 4, there is a method of operating an elongated medical assembly 102 having a distal-assembly tip 103 and an elongated puncture device 106 having a distal puncture tip 108. The method includes, and is not limited to, selectively maneuvering, at least in part, the distal-assembly tip 103, of the elongated medical assembly 102, along an assembly-tip movement path 404 terminating proximate to a first biological feature 911 (of a biological structure 902 of a patient 900). The method further includes, and is not limited to, selectively deploying, at least in part, the distal puncture tip 108, of the elongated puncture device 106, along a puncture-tip movement path 406 extending from the elongated medical assembly 102 and into, at least in part, the first biological feature 911, and then extending from the first biological feature 911 toward a second biological feature 912 of the patient 900.

Referring to the embodiment as depicted in FIG. 3, the elongated puncture device 106 is (preferably) selectively maneuverable (selectively extendable) from the elongated medical assembly 102 and/or selectively retractable toward the elongated medical assembly 102.

Referring to the embodiment as depicted in FIG. 3, the assembly-tip movement path 404 and the puncture-tip movement path 406 are configured to selectively intersect each other (one another). This is done, preferably, such that an obtuse angle 408 is subtended between the assembly-tip movement path 404 and the puncture-tip movement path 406.

Referring to the embodiment as depicted in FIG. 3, the elongated puncture device 106 is configured to provide (present) a first elongated member 301; this is preferably done once, or after, the elongated puncture device 106 is deployed or moved into, at least in part, (the interior of) the first biological feature 911 or the right coronary artery 921. The first elongated member 301 extends, at least in part, (laterally) along the right coronary artery 921; this is done, preferably, once (or after) the elongated puncture device 106 is deployed from the elongated medical assembly 102 (as depicted in FIG. 3). The valve leaflet 922 is positioned so that the valve leaflet 922 obstructs the right coronary artery 921 (as depicted in FIG. 3). The elongated puncture device 106 is also configured to provide (present) a second elongated member 302. The second elongated member 302 extends from the first elongated member 301 (located, at least in part, in the right coronary artery 921) toward the valve leaflet 922. The distal puncture tip 108 extends from the second elongated member 302 toward the valve leaflet 922; this is done (preferably) in such a way that the distal puncture tip 108 is in position to puncture through the valve leaflet 922 to form a puncture site 105 (a puncture hole, through the valve leaflet 922).

Referring to the embodiment as depicted in FIG. 3, the assembly-tip movement path 404 and the puncture-tip movement path 406 are configured to selectively intersect each other (one another). An obtuse angle 408 is subtended between the assembly-tip movement path 404 and the puncture-tip movement path 406; this is done, preferably, once or after (A) the elongated puncture device 106, in use, is selectively maneuvered (extended), at least in part, from the exit portal 104 of the elongated medical assembly 102, and (B) the distal puncture tip 108 is selectively maneuvered, at least in part, along the puncture-tip movement path 406.

Referring to the embodiment as depicted in FIG. 3, a bent section 410 is formed along the elongated puncture device 106 (once or after the assembly-tip movement path 404 and the puncture-tip movement path 406 are formed to subtend the obtuse angle 408 therebetween).

Preferably, the bent section 410 is formed within the first biological feature 911 while the elongated puncture device 106 travels along the assembly-tip movement path 404 through (at least in part) the first biological feature 911. In accordance with more preferred embodiment, the bent section 410 of the elongated puncture device 106 is formed before the bent section 410 enters the right coronary artery 921 of the heart 903 (for improved safety to the patient, and/or mitigation of inadvertent injury to the patient). The bent section 410 is positioned along the elongated puncture device 106, and the bent section 410 is configured to be inserted into the first biological feature 911.

Referring to the embodiments as depicted in FIG. 3 and FIG. 4, the distal puncture tip 108 is further maneuvered and positioned to protrude, at least in part, the puncture site 105 (as depicted in FIG. 3). As depicted in FIG. 4, the distal puncture tip 108 is configured (positioned) to be snared and pulled away from the puncture site 105 extending through the valve leaflet 922 (preferably, toward the elongated medical assembly 102). More specifically, the distal puncture tip 108 is positioned to be snared and pulled away from the puncture site 105 that is extended through the valve leaflet 922 (preferably extended toward the elongated medical assembly 102) after the distal puncture tip 108 is further maneuvered to protrude, at least in part, from the puncture site 105. It will be appreciated that maneuvering of the distal puncture tip 108 may include withdrawing the elongated puncture device 106 into the elongated medical assembly 102.

Referring to the embodiments as depicted in FIG. 3 and FIG. 4, the puncture-tip movement path 406 is, at least in part, spatially alignable (orientable, oriented) to face the assembly-tip movement path 404.

Referring to the embodiments as depicted in FIG. 3 and FIG. 4, the puncture-tip movement path 406 is, at least in part, spatially aligned (oriented) to intersect the assembly-tip movement path 404.

Referring to the embodiments as depicted in FIG. 3 and FIG. 4, the assembly-tip movement path 404 and the puncture-tip movement path 406 intersect orthogonally with each other.

Referring to the embodiment as depicted in FIG. 4, the elongated puncture device 106 is configured to form a puncture site 105 (also called a puncture hole). The puncture site 105 extends through the second biological feature 912; this is preferably done once the elongated puncture device 106 is deployed (maneuvered), at least in part, along the puncture-tip movement path 406, and the elongated puncture device 106 becomes located at (positioned proximate to) the second biological feature 912.

Referring to the embodiment as depicted in FIG. 4, the distal puncture tip 108 is configured to protrude and extend, at least in part, beyond the puncture site 105. The distal puncture tip 108 is also configured to be snared and pulled away from the puncture site 105 (preferably toward the elongated medical assembly 102) by a tip-snare device 500.

The tip-snare device 500 is configured to be maneuvered proximate to the distal puncture tip 108; this is preferably done in such a way that the tip-snare device 500, in use, snares (captures) and then moves the distal puncture tip 108 along a snare-retrieval path 502.

Referring to the embodiment as depicted in FIG. 5, the distal puncture tip 108 is configured to protrude and extend, at least in part, beyond the puncture site 105. The distal puncture tip 108 is also configured to be maneuvered away from the puncture site 105 (preferably toward the elongated medical assembly 102). For instance, the elongated puncture device 106 includes a memory shape metal, etc.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
an elongated medical assembly having a distal-assembly tip being, at least in part, selectively maneuverable along an assembly-tip movement path terminating proximate to a first biological feature of a biological structure of a patient; and
an elongated puncture device having a distal puncture tip being, at least in part, selectively deployable along a puncture-tip movement path extending from the elongated medical assembly and into, at least in part, the first biological feature, and then extending from the first biological feature toward a second biological feature of the patient, wherein said elongated puncture device is configured to form a puncture site extending through the second biological feature once the elongated puncture device is deployed, at least in part, along the puncture-tip movement path, and becomes located at the second biological feature.

2. The apparatus of claim 1, wherein:
the elongated puncture device is selectively maneuverable from, and selectively retractable toward, the elongated medical assembly.

3. The apparatus of claim 1, wherein:
the puncture-tip movement path is, at least in part, configured to be spatially aligned to face the assembly-tip movement path after deployment of the elongated puncture device.

4. The apparatus of claim 1, wherein:
the puncture-tip movement path is, at least in part, spatially aligned to intersect the assembly-tip movement path.

5. The apparatus of claim 1, wherein:
the assembly-tip movement path and the puncture-tip movement path intersect orthogonally with each other.

6. The apparatus of claim 1, wherein:
the assembly-tip movement path and the puncture-tip movement path are configured to selectively intersect each other, with an obtuse angle being subtended between the assembly-tip movement path and the puncture-tip movement path.

7. The apparatus of claim 1, wherein:
the assembly-tip movement path and the puncture-tip movement path are configured to selectively intersect each other, with an obtuse angle being subtended between the assembly-tip movement path and the puncture-tip movement path once the elongated puncture device, in use, is selectively maneuvered, at least in part, from an exit portal of the elongated medical assembly, and the distal puncture tip is selectively maneuvered, at least in part, along the puncture-tip movement path.

8. The apparatus of claim 1, wherein:
a bent section is positioned along the elongated puncture device; and
the bent section is configured to be inserted into the first biological feature.

9. The apparatus of claim 1, wherein:
the elongated medical assembly includes an exit portal, in which the assembly-tip movement path is extendable, at least in part, from the exit portal; and
the elongated puncture device is selectively maneuverable, at least in part, away from the exit portal of the elongated medical assembly.

10. The apparatus of claim 1, wherein:
the elongated puncture device includes a radio frequency wire with a radio frequency electrode fixedly positioned at the distal puncture tip.

11. The apparatus of claim 1, wherein:
the biological structure incudes the heart of the patient; and
the first biological feature includes the right coronary artery of the heart; and
the second biological feature includes a valve leaflet of the heart.

12. The apparatus of claim 1, wherein:
the elongated medical assembly includes:
a medical sheath assembly having a distal exit section leading to a lumen defined along, at least in part, a length of the medical sheath assembly; and
the elongated puncture device is configured to be receivable in, and movable, along,
at least in part, the lumen of the medical sheath assembly.

13. The apparatus of claim 1, wherein:
the elongated puncture device is configured to provide a first elongated member extending along the right coronary artery once the elongated puncture device is deployed from the elongated medical assembly.

14. The apparatus of claim 13, wherein:
the elongated puncture device is also configured to provide a second elongated member extending from the first elongated member located, at least in part, in the right coronary artery and toward the valve leaflet obstructing the right coronary artery, with the distal puncture tip extending from the second elongated member toward the valve leaflet in such a way that the distal puncture tip is positioned to puncture through the valve leaflet to form a puncture site through the valve leaflet.

15. The apparatus of claim 13, wherein:
the distal puncture tip is positioned to be snared and pulled away from a puncture site that extends through the valve leaflet and toward the elongated medical assembly after the distal puncture tip is further maneuvered to protrude, at least in part, from the puncture site.

16. The apparatus of claim 1, wherein:
the distal puncture tip is configured to protrude and extend, at least in part, beyond a puncture site, and is also configured to be maneuvered away from the puncture site.

17. The apparatus of claim 1, wherein:
the distal puncture tip is configured to protrude and extend, at least in part, beyond a puncture site, and is also configured to be snared and pulled away from the puncture site by a tip-snare device, in which the tip-snare device is configured to be maneuvered proximate to the distal puncture tip in such a way that the tip-snare device, in use, snares and then moves the distal puncture tip.

18. The apparatus of claim 1, wherein:
the elongated puncture device includes:
a first cutting element positioned at the distal puncture tip, and the first cutting element being configured to form a puncture site through the second biological feature; and
a second cutting element being spaced apart from the first cutting element, and the second cutting element being configured to form a laceration along, at least in part, the second biological feature starting from the puncture site after the first cutting element, in use, forms the puncture site through the second biological feature.

19. An apparatus for an elongated medical assembly having a distal-assembly tip being, at least in part, selectively maneuverable along an assembly-tip movement path terminating proximate to a first biological feature of a biological structure of a patient, and the apparatus comprising:
an elongated puncture device having a distal puncture tip; and
the distal puncture tip being, at least in part, selectively deployable along a puncture-tip movement path extending from the elongated medical assembly and into, at least in part, the first biological feature, and then extending from the first biological feature toward a second biological feature of the patient.

20. A method of operating an elongated medical assembly having a distal-assembly tip and an elongated puncture device having a distal puncture tip, the method comprising:
selectively maneuvering, at least in part, the distal-assembly tip, of the elongated medical assembly, along an assembly-tip movement path terminating proximate to a first biological feature of a biological structure of a patient; and
selectively deploying, at least in part, the distal puncture tip, of the elongated puncture device, along a puncture-tip movement path extending from the elongated medical assembly and into, at least in part, the first biological feature, and then extending from the first biological feature toward a second biological feature of the patient.
